(19) United States
(12) Patent Application Publication
(10) Pub. No.: US 2023/0160791 A1
(43) Pub. Date: May 25, 2023

(54) DEVICE AND METHOD FOR TISSUE PROCESSING

(71) Applicants: Camillo Ricordi, Miami, FL (US); Giacomo Lanzoni, Miami, FL (US); University of Miami, Miami, FL (US)

(72) Inventors: Camillo Ricordi, Miami, FL (US); Giacomo Lanzoni, Miami, FL (US)

(21) Appl. No.: 17/916,969

(22) PCT Filed: Apr. 6, 2021
(86) PCT No.: PCT/US21/25941

§ 371 Date: Oct. 4, 2022

Related U.S. Application Data
(60) Provisional application No. 63/005,900, filed on Apr. 6, 2020.

(51) Int. Cl. G01N 1/28

(52) U.S. Cl. CPC G01N 1/286; G01N 2001/2873

(57) ABSTRACT

A tissue processing chamber is disclosed. The tissue processing chamber includes at least one rotary blade housed within a tissue chamber and a drive shaft coupled to the rotary blades. Rotation of the drive shaft rotates the rotary blades and presses a tissue sample through a screen adjacent to the at least one rotary blade, wherein rotation of the at least one rotary blade presses processed tissue through the screen. The tissue processing device also includes a collection chamber coupled to the tissue chamber configured to collect the processed tissue.

### CROSS-REFERENCE TO RELATED APPLICATIONS

This application is a 35 U.S.C. 371 National Stage of International Application No. PCT/US21/25941 filed Apr. 6, 2021, which claims priority to U.S. Provisional Application No. 63/005,900, filed Apr. 6, 2020, the contents of which are hereby incorporated by reference in their entirety for all intents and purposes.

### FIELD OF THE DISCLOSURE

This disclosure relates generally to an apparatus and method for processing tissue. More specifically, this disclosure relates to isolating tissue, for example, from tissue samples or organs by way of a tissue processing chamber.

### BACKGROUND

Many different methods and approaches have been attempted to isolate individual cells from their respective parent organs or larger tissue samples. Prior methods have produced isolated cells with some cell destruction. This cell destruction can result from the relatively severe mechanical stimulation that is used to isolate cells from an organ. Additionally, many known methods require addition of an enzyme to break down the tissue samples.

The disadvantages of mechanical and enzymatic methods for individual cell isolation from parent organs or tissues known in the art has resulted in a need in the art for more effective devices and methods for individual cell isolation from parent organs or tissues that provides greater yields of a greater percentage of intact, viable cells.

### SUMMARY

Provided herein are devices and methods of use thereof for individual cell isolation from parent organs or tissues that provides greater yields of a greater percentage of intact, viable cells.

One aspect of the disclosure is a tissue processing device. The tissue processing device includes a tissue chamber. The tissue chamber includes at least one rotary blade housed within the tissue chamber, a drive shaft coupled to the at least one rotary blade, wherein rotation of the drive shaft is configured to rotate the at least one rotary blade, and a screen adjacent to the rotary blades, wherein rotation of the at least one rotary blade is configured to press processed tissue of a tissue sample through the screen. The tissue processing device further includes a collection chamber coupled to the tissue chamber configured to collect the processed tissue.

In another aspect, a tissue processing system is disclosed. The tissue processing system includes a tissue chamber. The tissue processing chamber includes at least one rotary blade housed within the tissue chamber, a drive shaft coupled to the at least one rotary blade, wherein rotation of the drive shaft is configured to rotate the at least one rotary blade, and wherein a distal end of the drive shaft comprises a motor coupling, and a screen adjacent to the at least one rotary blade, wherein rotation of the at least one rotary blade is configured to press processed tissue of a tissue sample through the screen. The tissue processing system further includes a collection chamber coupled to the tissue chamber configured to collect the processed tissue and an isolation chamber coupled to the tissue chamber and the collection chamber. The isolation chamber includes a motor coupled to the motor coupling configured to rotate the drive shaft

In another aspect, a tissue processing method is disclosed. The tissue processing method includes rotating at least one rotary blade within a tissue chamber. The tissue processing method additionally includes pressing at least a portion of a tissue sample through a screen adjacent to the at least one rotary blade via impeller forces of the at least one rotary blade. The tissue processing method further includes collecting processed tissue in a collection chamber.

These and other features and advantages of the invention disclosed herein will be more fully understood from the following detailed description taken together with the accompanying drawings and the claims. It is noted that the scope of the claims is defined by the recitations therein and not by the specific discussion of features and advantages set forth in the present description.

### BRIEF DESCRIPTION OF THE FIGURES

The above, as well as additional, features will be better understood through the following illustrative and non-limiting detailed description of example embodiments, with reference to the appended drawings.

FIG. 1A illustrates a cross-sectional view of an example tissue processing chamber, according to an example embodiment.

FIG. 1B illustrates a cross-sectional view of an example tissue processing chamber, according to an example embodiment.

FIG. 2 illustrates another cross-sectional view of an example tissue processing chamber, according to an example embodiment.

FIG. 3 illustrates a schematic of an example tissue processing system, according to an example embodiment.

FIG. 4 illustrates a schematic of an example tissue processing system and an example infusion bag, according to an example embodiment.

FIG. 5A illustrates an exploded view of an example tissue processing chamber, according to an example embodiment.

FIG. 5B illustrates an exploded view of an example tissue processing chamber, according to an example embodiment.

FIG. 5B illustrates an exploded view of an example tissue processing chamber, according to an example embodiment.

FIG. 6 is a flow chart illustrating an example method of the present disclosure.

FIG. 7A illustrates an example drive shaft and rotary blades, according to an example embodiment.

FIG. 7B illustrates an example drive shaft and rotary blades, according to an example embodiment.

FIG. 8 illustrates an example screen, according to an example embodiment.

FIG. 9 illustrates an example detachable stand, according to an example embodiment.

FIG. 10 illustrates an example tissue loading port cap, according to an example embodiment.

All the figures are schematic, not necessarily to scale, and generally only show parts which are necessary to elucidate example embodiments, wherein other parts can be omitted or merely suggested.

### DETAILED DESCRIPTION

Example embodiments are now described more fully hereinafter with reference to the accompanying drawings. That which is encompassed by the claims can, however, be embodied in many different forms and should not be construed as limited to the embodiments set forth herein; rather, these embodiments are provided by way of example. Furthermore, like numbers refer to the same or similar elements or components throughout.

In accordance with the principles herein, a tissue processing chamber, shown generally at 100, provides processing and separation of tissue samples from larger tissue samples or organs. The tissue processing chamber can include rotary blades which, through impeller forces of the rotating rotary blades, press the larger tissue sample through a screen into a collection chamber. Processed tissue can then be extracted from the collection chamber, for example, for testing, culture, or clinical use.

For example, tissues that can be processed include, but are not limited to mesodermal-derived, endodermal-derived, ectodermal derived tissues, extraembryonic and fetal adnexa tissues, adipose tissue, pancreatic tissue, liver tissue, biliary tree issue, intestinal tissue, lung tissue, kidney tissue, bone tissue, bone marrow tissue, cartilage, muscle tissue, tendon, ligaments, amniotic tissue, chorionic tissue, umbilical cord tissue, placenta, blood vessels tissue, ovarian tissue, endocrine tissue, thyroid gland tissue, parathyroid gland tissue, adrenal gland tissue, pituitary gland tissue, pineal gland tissue, thymic tissue, dermal tissue, epidermal tissue, connective tissue, fibrous tissue, and central and peripheral nervous tissue. Tissue processing can be performed by activating the impeller at a specific rotational speed, or at a range of speeds, in clockwise or counter-clockwise direction. The geometry of the blade and of the screen can be modified to yield tissue fragments with different shapes. Screens of different geometries can be replaced in the same instrument to yield tissue fragments of different sizes. Instruments connected in series and loaded with screens of progressively smaller sizes can process tissue yielding fragments of decreasing size throughout the series.

Further, testing can include, but is not limited to, measurement of the size, volume, and number of tissue fragments via imaging, measurement of the weight of the fragments via mechanical scale or balance, measurement of electrical impedance of tissue fragments, suspended in an electrolyte, when passing through an aperture between electrodes (Coulter method, Coulter principle), measurement of viability of tissue fragments via staining and imaging, analysis of RNA expression and gene expression via northern blotting, hybridization, fluorescent in situ hybridization, reverse transcription-Polymerase Chain Reaction (RT-PCR), quantitative RT-PCR, microarray, Tiling array, next-generation sequencing, RNA sequencing, analysis of DNA content via DNA sequencing, analysis of protein expression via Liquid Chromatography—Tandem Mass Spectrometry, Gas Chromatography, analysis of immunomodulatory function, analysis of hormone-release function, and/or analysis of the release of factors in the fluid milieu via sensors.

In some example embodiments, culture that can be done with samples include tissue fragments can be cultured with culture media to generate organotypic cultures; tissue fragments can be cultured alone or in combination with other cells, tissue fragments, tissues, or matrices; tissue fragments can be cultured with culture media in static culture, in agitation, in perifusion (i.e., fluid flow), in an automated bioreactor system; and/or in two-dimensional or three-dimensional culture conditions, or in a compartimentalized device. Tissue fragments can be cultured in culture media in non-adherent conditions, in adherent conditions, or in embedded conditions (such as in a matrix or material); tissue fragments can be maintained in a liquid medium, or at the liquid-gas interface; tissue fragments can be suspended in cryopreservation medium and subsequently cryopreserved, can be cryopreserved directly, can be lyophilized, can be maintained in hypothermic conditions, or can be encapsulated.

In some example embodiments, clinical uses for the samples include, but are not limited to, manipulation of the tissue via mechanical processing of tissue into tissue fragments, in the presence or absence of washing, concentration, and/or preservation steps. Minimally manipulated tissue can be utilized for homologous use and can be utilized clinically in the autologous setting, or in the allogeneic setting. Processed tissue fragments can be implanted for homologous use (i.e., for repair, reconstruction, replacement, or supplementation of a recipient's cells or tissues). In homologous uses, fragments of tissue can perform the same basic function or functions in the recipient as in the donor. Human tissues undergoing minimal manipulation and intended for application in homologous uses are currently classified as human cellular or tissue product (HCT/P). Adipose tissue fragments can be utilized in plastic surgery, musculoskeletal, reparative (traumatic lesions, burns and wounds) regenerative medicine applications, and reconstructive surgery applications. Cartilage tissue fragments can be utilized in reconstructive and orthopedic surgery application to replace cartilage after fracture, loss, or disease. Stromal Vascular Tissue Fragments can be utilized in reconstructive surgery applications. Endocrine tissue fragments can be used to functionally replace or supplement the endocrine tissue of the recipient. Optionally, processed tissue fragments can be cultured and/or cryopreserved, before clinical use.

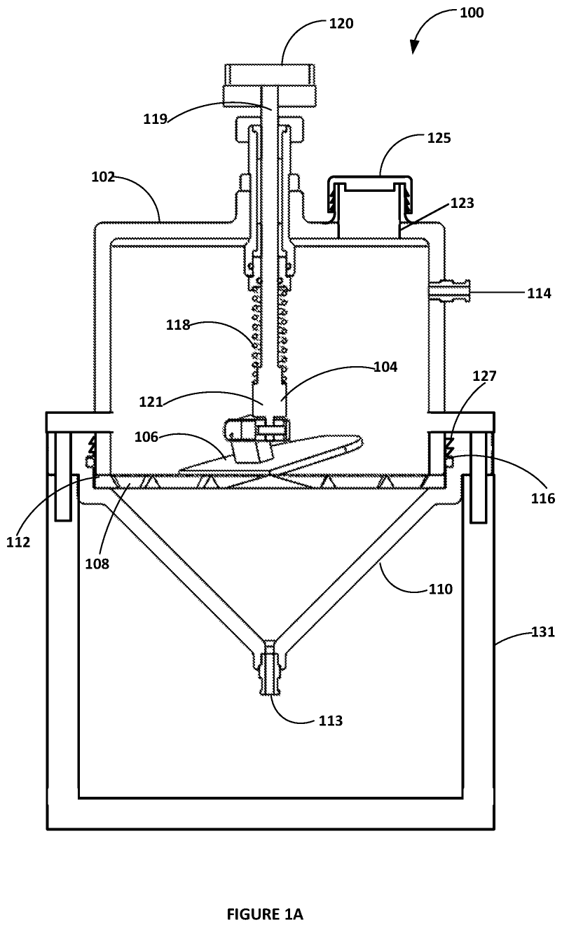

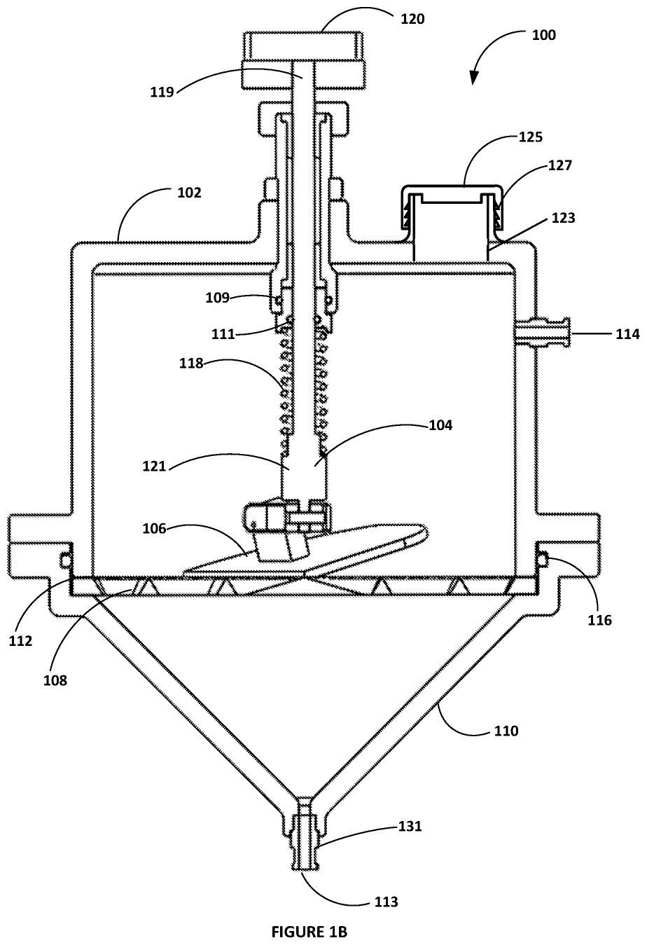

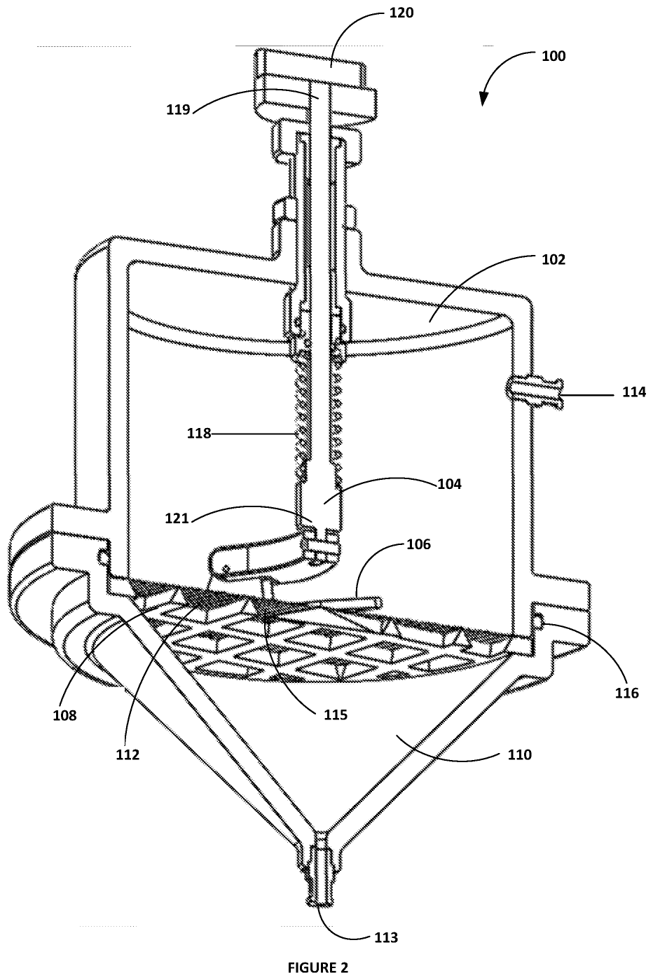

Now referring to FIGS. 1A-2, schematic cross-sectional views of an example tissue processing chamber 100, according to an example embodiment are shown. A tissue processing chamber 100 includes a tissue chamber 102, a drive shaft 104, one or more rotary blades 106, a support grid 108, a collection chamber 110, a screen 112, and, in some examples, a detachable stand 131.

In example embodiments, the tissue chamber 102 can be cylindrical, or substantially cylindrical, and house a portion of the drive shaft 104, the rotary blades 106, the support grid 108, and the screen 112. More specifically, the tissue chamber 102 can be a vessel defined by an outer boundary and a space within the outer boundary. The space within the outer boundary can have any useful and convenient shape. Example configurations include cylindrical (as shown in FIGS. 1A-2), spherical, or conical shapes, among many others.

In some examples, the tissue chamber 102 can be constructed of an autoclavable material. Autoclavable material can withstand the pressure and temperature of tissue processing, as well as repeated sterilization. For example, the tissue chamber 102 can comprise a high grade polymer material. This is desirable, as tissue processing requires regulated temperatures and pressures. It should be understood that other materials and example configurations of the tissue chamber 102 are possible.

The tissue chamber 102 includes an inlet for depositing a tissue sample, such as a tissue loading port 123. The tissue loading port 123 can include a tissue loading port cap 125. The tissue loading port 123 can be configured such that, during use, the tissue chamber 102 can be assembled and sterilized before a tissue sample is added. The tissue sample can then be added by removing the tissue loading port cap 125 and depositing the tissue sample into the tissue chamber 102. In some example, the tissue loading port 123 and tissue loading port cap 123 can fasten to each other by way of a threaded connection, however other example embodiments are possible.

Similar to the tissue chamber 102, in some examples, the tissue loading port 123 and tissue loading port cap 125 can include autoclavable material, such as a high grade polymer material. Additionally or alternatively, the tissue loading port 123 and tissue loading port cap 125 can include material that can be sterilized via irradiation or via gas sterilization. It should be understood that other materials and example configurations of the tissue loading port 123 and tissue loading port cap 125 are possible.

Additionally or alternatively, the tissue sample can be deposited into the tissue chamber 102 directly, for example, from a top portion of the tissue chamber. In an alternative embodiment, the tissue sample can be deposited into the tissue chamber 102 before the tissue chamber 102 is coupled to the collection chamber 110. For example, the tissue chamber 102 and collection chamber 110 can include a threaded connection 127 for deposit and removal of the tissue sample, as shown in FIG. 1A. The threaded connection 127 allows for deposit and removal of the tissue sample from the tissue chamber 102.

Additionally or alternatively, the tissue sample can be deposited by way of an inlet, such as a luer lock 114, or equivalent. The luer lock 114 can include fluid fittings used for making leak-free, sterile connections between a male-taper fitting and its mating female part on the tissue chamber 102. The luer lock 114 can couple to an inlet tube (not shown) to deposit a specimen, such as homogenate, or saline into the tissue chamber 102. Additionally, or alternatively, in some examples, the inlet tube coupled to the luer lock 114 can deposit saline into the tissue chamber 102. Many other examples of alternative locks or inlets are possible.

The drive shaft 104 can be an elongated rod at least partially housed by the tissue chamber 102 and extending vertically, or substantially vertically, through the tissue chamber 102. Additionally, in some embodiments, the drive shaft 104 includes a motor coupling 120 on a distal end 119 and the rotary blades 106 on a proximal end 121.

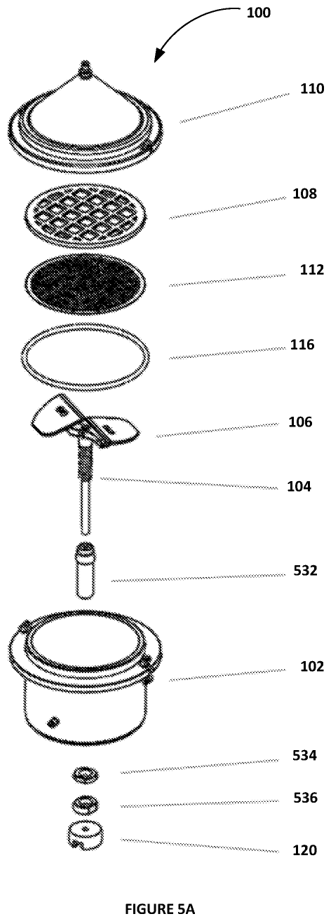

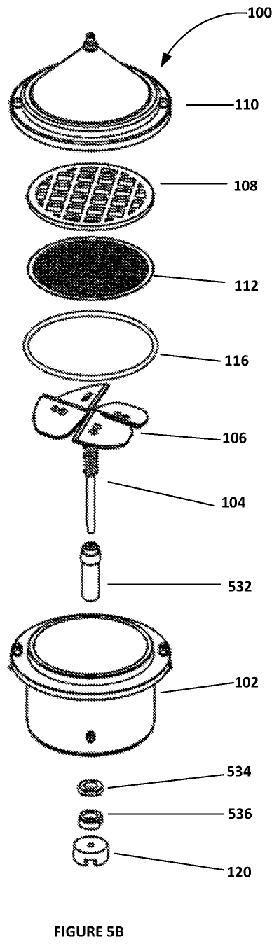

The motor coupling 120 can be coupled to a motor on an isolation chamber (shown in FIGS. 5A-5C). In practice, operation of the motor rotates the drive shaft 104 and the rotary blades 106 about a vertical axis (i.e., the axis along the drive shaft 104).

Further, in some example embodiments, the drive shaft 104 includes a compression spring 118. The compression spring 118 can surround or substantially surround the drive shaft 104 and allow vertical movement of the rotary blades 106 along the drive shaft 104. The compression spring 118 can push the rotary blades 106 in position against the screen 112, while allowing the rotary blades 106 to adjust position along the drive shaft 104 and surpass potential blockages. Accordingly, large tissue chunks are progressively pushed through the openings of the screen 112, and the rotary blades 106 will not get stuck or stopped by large tissue chunks. In some embodiments, it is possible to adjust the compression force of that the rotary blades 106 apply to the tissue sample against the screen 112 with the spring tension adjustment nut 534 and lock nut 536 (as shown in FIGS. 5A-5C).

Additionally, in some examples, the drive shaft 104 and the rotary blades 106 can be configured to rotate in both clockwise and counter-clockwise directions.

The one or more rotary blades 106 are adjacent to the screen 112 and, in some examples, include stainless steel or another non-corrosive metal. Impeller forces of the rotating rotary blades 106 press the tissue sample through the screen 112 to process and break down the tissue sample into smaller pieces. In practice, rotation of the rotary blades 106 presses the tissue sample through the screen 112. Pressing the tissue sample through the screen 112, via the rotary blades 106, in this manner can be done in a sterile, full-immersion system to minimize tissue trauma.

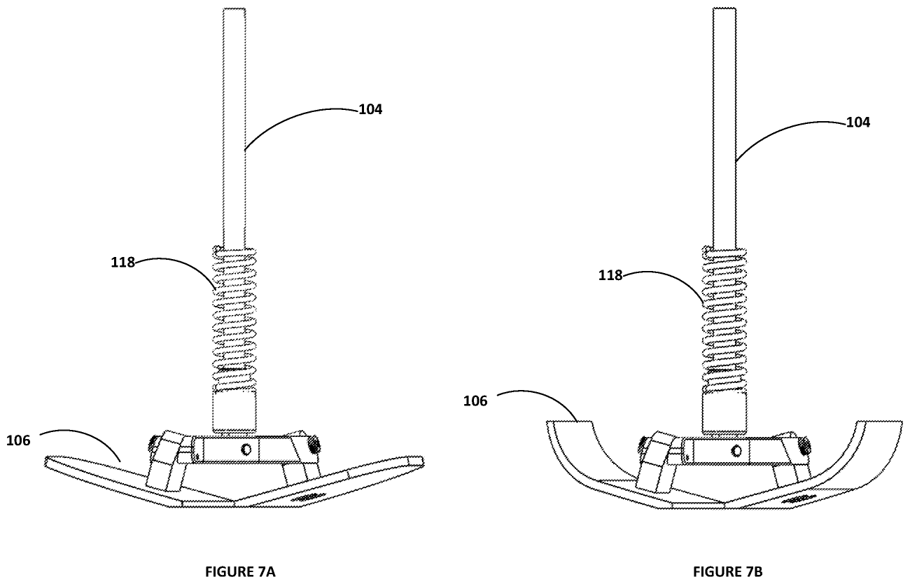

In some examples, the tissue processing chamber 100 can include two rotary blades 106, as shown in FIGS. 1A and 1B. In alternative embodiments, the tissue processing chamber 100 can include one blade or three or more rotary blades 106. Further, a variety of shapes and sizes of rotary blades 106 can be used in different embodiments. Many examples and configurations of rotary blades 106 are possible, such as those shown in FIGS. 7A-7B.

In some example embodiments, the rotary blades 106 can additionally be configured to pivot or rotate about a horizontal axis to facilitate various sizes, shapes, and consistency of different tissue samples.

The screen 112 can, for example, be a wire mesh. In some examples, the wire mesh can include non-corrosive metal, such as stainless steel, which is desirable, as the screen 112 must withstand repeated sterilization.

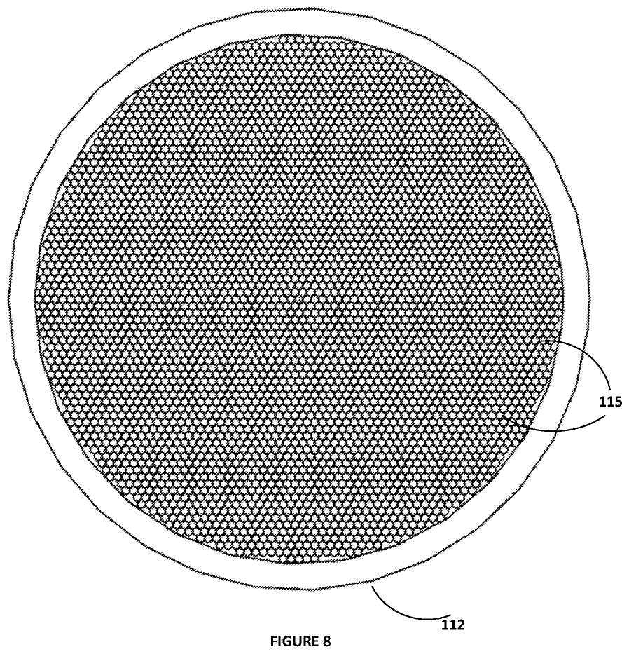

The screen 112 includes a plurality of pores 115 for the tissue sample to be pressed through. In some examples, pores 115 can be hexagonal (as shown in FIG. 8) or round in shape. Many other pore shapes and configurations are possible. The desired pore size can vary depending on the desired size of the processed tissue. For example, in some embodiments, it can be desirable to break down a tissue sample into very fine pieces. In these examples, the pore size of the screen 112 can be very small. Alternatively, it can be desirable to break down a tissue sample in larger pieces. In these examples, pore sizes of the screen 112 can be larger. In some examples, pore sizes can range from 20 μm to 3 mm.

Further, in some examples, the screen 112 can be removable and/or interchangeable such that the tissue processing chamber 100 can use a variety of different screens.

The support grid 108 is adjacent to and supports the screen 112. In some examples, the support grid 108 can also include pores 115. The pores of the support grid 108 can be larger than the pores of the screen 112. In practice, impeller forces of the rotating rotary blades 106 press the processed tissue through the support grid 108, in addition to the screen 112, to enter the collection chamber 110.

The collection chamber 110 is coupled to the tissue chamber 102 adjacent to the screen 112 and the support grid 108. In some examples, the collection chamber 110 can be conical. Many other shapes and configurations of the collection chamber 110 are possible.

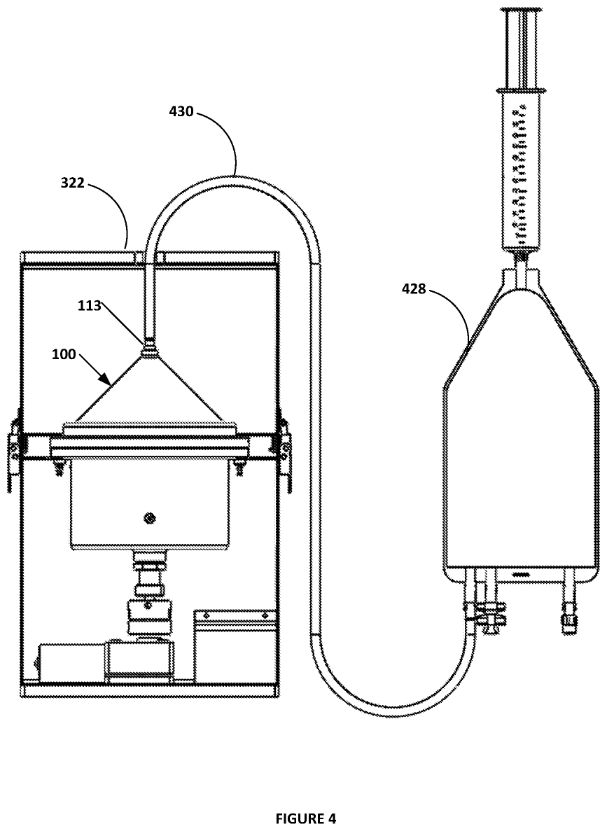

Further, in example embodiments, the collection chamber 110 also includes an outlet 113 for outflow of processed tissue. The outlet 113 can attach to a sterile collection bag (not shown). Additionally or alternatively, the outlet 113 can attach to an outlet tube (as shown in FIG. 4).

In some examples, the collection chamber 110 can be constructed of an autoclavable material (i.e., material that can withstand the pressure and temperature of tissue processing). For example, the collection chamber 110 can comprise a high grade polymer material. This is desirable, as tissue processing requires regulated temperatures and pressures.

Further, in some example embodiments, the tissue processing chamber 100 can include an O-ring seal 116 between the tissue chamber 102 and the collection chamber 110. In some examples, the O-ring seal 116 can create a static hermetic seal.

Additionally or alternatively, in some examples, the tissue chamber 100 can include two or more additional O-rings 109 and 111. These additional O-rings 109 and 111 can create a dynamic seal around the drive shaft 104.

In practice, specimen (e.g., a tissue sample) is deposited into the tissue chamber 102 via an inlet (e.g., the luer lock 114). A motor then spins the drive shaft 104 about a vertical (or substantially vertical) axis rotating the rotary blades 106. The rotating rotary blades 106 press the tissue through the through the screen 112, breaking down the processed tissue. Once the tissue passes through the screen 112, tissue collects in the collection chamber 110. In the example configuration shown in FIGS. 1A-2, the tissue sample is pressed downwards through the screen 112 into the collection chamber 110.

In an alternate embodiment, the tissue processing chamber 100 is configured with the tissue chamber 102 below the collection chamber 110 (i.e., the tissue processing chamber 100 can be inverted or flipped upside down). This configuration is desirable in examples where the tissue sample includes a lipid. Namely, in practice, the lipids will float or rise to the top of the tissue chamber 102. Impeller forces of the rotary blades 106 will press the lipids through the screen 112 and into the collection chamber 110.

In some examples, the tissue processing chamber 100 can include a detachable stand 131. The detachable stand 131 can be configured to detachably fasten to the tissue chamber 102, as shown in FIG. 1A. In practice, the detachable stand 131 can be utilized to hold the tissue processing chamber 100 while loading the tissue sample. In some examples, the detachable stand 131 includes autoclavable material and/or material that can be sterilized via irradiation or gas sterilization, such as high grade polypropylene or aluminum. Many other shapes and materials may be utilized for the detachable stand 131.

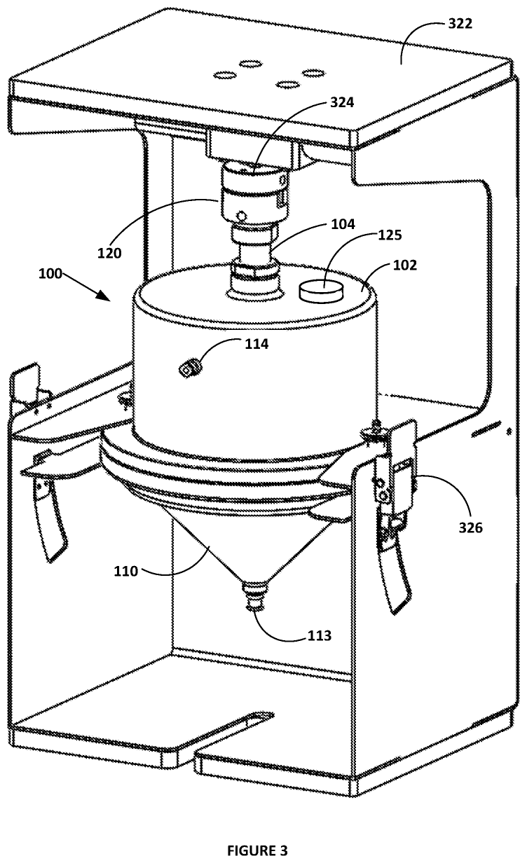

Now referring to FIG. 3, a tissue processing chamber 100 shown in an isolation chamber 322. The tissue processing chamber 100 can be coupled to isolation chamber 322 during operation. For example, the motor coupling 120 can couple to a motor 324 by a latch and/or lock (not shown) on the motor 324. In practice, when the motor coupling 120 is coupled to the motor 324, operation of the motor 324 rotates the drive shaft 104 and rotary blades 106. Further, in some example embodiments, the motor 324 can be configured to rotate the drive shaft 104 and the rotary blades 106 in both a clockwise direction and a counter clockwise direction about a vertical axis.

The motor 324 can be configured to be on either the top or the bottom of the isolation chamber 322 to accommodate for different configurations of the tissue processing chamber 100. For example, as shown in FIG. 3 the motor 324 can be coupled to the top portion of the isolation chamber 322 in examples where the tissue chamber 102 is above the collection chamber 110. Alternatively, the motor 324 can be coupled to a bottom portion of the isolation chamber 322 in examples where the tissue chamber 102 is below the collection chamber 110 (e.g., in embodiments where the tissue sample includes lipids). Additionally or alternatively, the isolation chamber 322 can be inverted (i.e., flipped upside down) to accommodate for different tissue processing chamber 100 configurations and/or tissue samples.

Additionally, portions of the tissue chamber 102, collection chamber 110, and/or O-ring seal 116 can couple to the isolation chamber 322. In some examples, the isolation chamber 322 can include locks 326 to stabilize the tissue processing chamber 100 during operation. It should be understood that any known type of connection mechanism can be used to attach the tissue processing chamber to the isolation chamber.

Now referring to FIG. 4, the tissue processing chamber 100 coupled to an infusion bag 428, according to an example embodiment. In some example embodiments, the outlet 113 of the collection chamber 110 can be coupled to one end of an outlet tube 430. The opposite end of the outlet tube 430 can be coupled to the infusion bag 428. Additionally or alternatively, in some examples, the outlet 113 can be coupled directly to the infusion bag 428, or an alternate sterile collection bag. This example configuration can be desirable in embodiments where the tissue chamber 102 is on top of the collection chamber 110 (as shown in FIGS. 1A-2, for example). Other known methods of extracting the tissue sample from the collection chamber 110 can be utilized, such as collection with a syringe, pumping via tubing and pump, or disassembling the chamber and pouring the content of the collection chamber.

Now referring to FIGS. 5A-5C, an exploded view of an example tissue processing chamber 100, according to an example embodiment. The example tissue processing chamber 100 includes all the components as shown in FIGS. 1A-4 and described above. In some example embodiments, the tissue processing chamber 100 can additionally include a threaded adaptor with rotating seals 532. Further, the tissue processing chamber 100 can also include a spring tension adjustment nut 534 and a lock nut 536. In practice, the tension adjustment nut 534 and lock nut 536 allow adjustment of the compression force the rotary blades 106 apply to the tissue sample against the screen 112. Other mechanical fittings and configurations are possible and can be utilized.

Further, in some example embodiments, the tissue processing chamber 100 can include two rotary blades 106, as shown in FIG. 5A. Alternatively, the tissue processing chamber 100 can include four rotary blades 106 or one rotary blade 106, as shown in FIG. 5B and FIG. 5C, respectively.

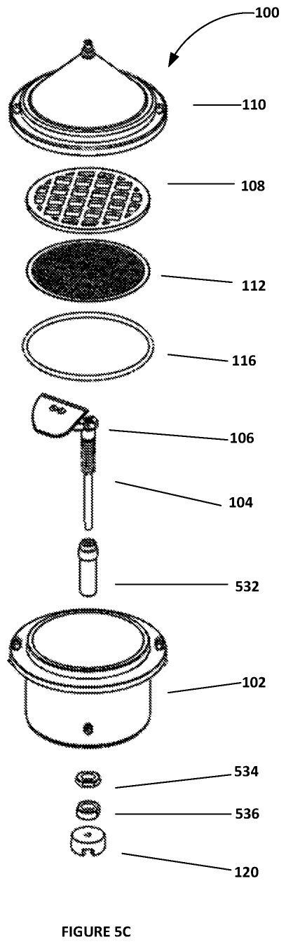

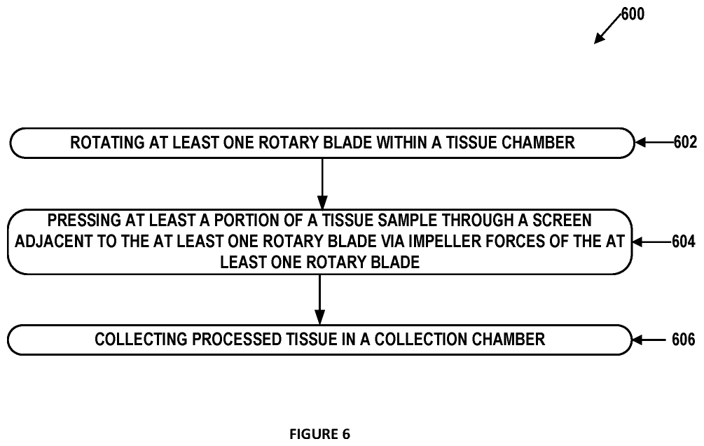

Now referring to FIG. 6, a flow chart illustrating an example method 900 of the present disclosure. Each block or portions of each block in FIG. 6, and within other processes and methods disclosed herein, can be performed by or in accordance with the tissue processing chamber described above with respect to FIGS. 1A-5C. Alternative implementations are included within the scope of the examples of the present disclosure in which functions can be executed out of order from that shown or discussed, including substantially concurrent or in reverse order, depending on the functionality involved, as would be understood by those reasonably skilled in the art.

Method 600 begins at block 602, which involves rotating at least one rotary blade within a tissue chamber.

At block 604, method 600 involves pressing at least a portion of a tissue sample through a screen adjacent to the at least one rotary blade via impeller forces of the at least one rotary blade. In some embodiments, before block 604, the method further involves depositing the tissue sample into the tissue chamber by way of a luer lock on the tissue chamber. Additionally, in some embodiments, the tissue chamber comprises a support grid adjacent to the screen and wherein the method further comprises pressing the processed tissue through the support grid.

At block 606, method 600 involves collecting processed tissue in a collection chamber. In some embodiments, method 600 further involves extracting the processed tissue from the collection chamber via a sterile collection bag attached to an outlet of the collection chamber.

Additionally, in some embodiments, method 600 can further involve depositing saline into the tissue chamber by way of a luer lock on the tissue chamber.

Now referring to FIGS. 7A and 7B, example configurations of rotary blades 106 are shown. A variety of shapes and sizes of rotary blades 106 can be used in different embodiments. For example, the rotary blades 106 can be flat, as shown in FIG. 7A. Alternatively, the rotary blades 106 can be curved, as shown in FIG. 7B. Many other examples are possible.

Now referring to FIG. 8, a screen 112, according to an example embodiment is shown. In some example embodiments, the pores 115 of the screen 112 can be hexagonal in shape, as shown in FIG. 8. Alternatively, the pores 115 can be round or oval in shape. Many other examples of shapes and sizes of screens 112 and pores 115 are possible.

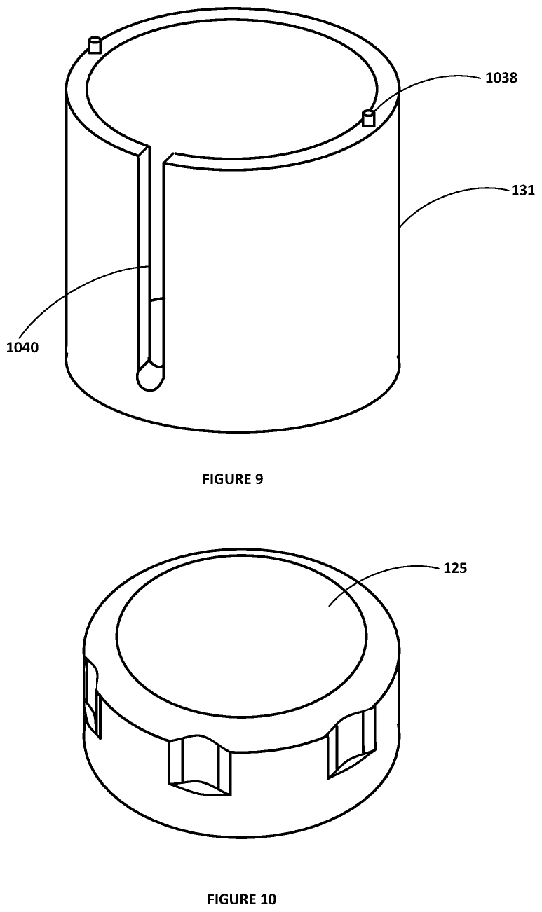

Now referring to FIG. 9, an example detachable stand 131, according to an example embodiment, is shown. As noted above, the detachable stand 131 is configured to detachably fasten to the tissue chamber 102, as shown in FIG. 1A, and can be utilized to hold the tissue processing chamber 100 while loading the tissue sample. Additionally or alternatively, the detachable stand 131 can be used to hold the tissue chamber 102 while loading the tissue sample. The detachable stand 131 can include one or more notches 1038 compatible with corresponding holes (not shown) of the tissue chamber 102. Further, in some examples, the detachable stand 131 can include a slit 1040 to accommodate tubing, such as outlet tube 430, shown in FIG. 4. Many other examples of shapes and sizes of detachable stands 131 are possible. For example, different shapes and geometries can be generated to interlock the detachable stand 131 and the tissue processing chamber 100.

Now referring to FIG. 10, an example tissue loading port cap 125, according to an example embodiment is shown. In practice, the tissue sample can be added by removing the tissue loading port cap 125 and depositing the tissue sample into the tissue chamber 102. The tissue loading port 123 and tissue loading port cap 123 can fasten to each other by way of a threaded connection, however other example connection types are possible. Many other examples of shapes and sizes of the tissue loading port cap 123 are possible.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

What is claimed is:

1. A tissue processing device comprising:
a tissue chamber comprising:
at least one rotary blade housed within the tissue chamber;
a drive shaft coupled to the at least one rotary blade, wherein rotation of the drive shaft is configured to rotate the at least one rotary blade; and
a screen adjacent to the rotary blades, wherein rotation of the at least one rotary blade is configured to press processed tissue of a tissue sample through the screen; and
a collection chamber coupled to the tissue chamber configured to collect the processed tissue from the tissue chamber.

2. The tissue processing device of claim 1, wherein the tissue chamber comprises a luer lock configured to deposit the tissue sample into the tissue chamber.

3. The tissue processing device of claim 1, wherein the tissue chamber is made of autoclavable material.

4. The tissue processing device of claim 1, wherein the collection chamber is made of autoclavable material.

5. The tissue processing device of claim 1, wherein the screen comprises a plurality of pores, and wherein the plurality of pores are between 20 μm to 3 mm.

6. The tissue processing device of claim 1, further comprising a support grid adjacent to the screen, wherein the support grid comprises a plurality of pores.

7. The tissue processing device of claim 1, wherein the at least one rotary blade is configured to rotate in both a clockwise direction and a counter clockwise direction.

8. The tissue processing device of claim 1, wherein the at least one rotary blade comprises a plurality of rotary blades.

9. The tissue processing device of claim 1, wherein the collection chamber further comprises an outlet coupled to a sterile collection bag or to an outlet tube.

10. The tissue processing device of claim 1, wherein the tissue sample comprises lipids, wherein the lipids rise to a top portion of the tissue chamber to the at least one rotary blade, and wherein rotation of the at least one rotary blade is configured to press processed tissue through the screen.

11. The tissue processing device of claim 1 wherein a distal end of the drive shaft extends through an end of the tissue chamber, wherein the distal end of the drive shaft comprises a motor coupling.

12. The tissue processing device of claim 12, wherein the tissue processing device further comprises an isolation chamber that houses the tissue chamber and the collection chamber.

13. The tissue processing device of claim 13, wherein the isolation chamber comprises:
a motor coupled to the motor coupling, wherein operation of the motor rotates the drive shaft and the at least one rotary blade.

14. A tissue processing system comprising:
a tissue chamber comprising:
at least one rotary blade housed within the tissue chamber;
a drive shaft coupled to the at least one rotary blade, wherein rotation of the drive shaft is configured to rotate the at least one rotary blade, and wherein a distal end of the drive shaft comprises a motor coupling; and
a screen adjacent to the at least one rotary blade, wherein rotation of the at least one rotary blade is configured to press processed tissue of a tissue sample through the screen;
a collection chamber coupled to the tissue chamber configured to collect the processed tissue; and
an isolation chamber coupled to the tissue chamber and the collection chamber, the isolation chamber comprising:
a motor coupled to the motor coupling configured to rotate the drive shaft.

15. The tissue processing system of claim 15, wherein the at least one rotary blade comprises a plurality of rotary blades.

16. A method for processing tissue comprising:
rotating at least one rotary blade within a tissue chamber;
pressing at least a portion of a tissue sample through a screen adjacent to the at least one rotary blade via impeller forces of the at least one rotary blade; and
collecting processed tissue in a collection chamber.

17. The method of claim 18, further comprising:
depositing the tissue sample into the tissue chamber through a luer lock on the tissue chamber.

18. The method of claim 18, further comprising:
depositing saline into the tissue chamber through a luer lock on the tissue chamber.

19. The method of claim 18, further comprising:
extracting the processed tissue from the collection chamber via a sterile collection bag attached to an outlet of the collection chamber.

20. The method of claim 18, wherein the tissue chamber comprises a support grid adjacent to the screen and wherein the method further comprises:
pressing the processed tissue through the support grid.
